(19) United States
(12) Patent Application Publication
(10) Pub. No.: US 2012/0053411 A1
(43) Pub. Date: Mar. 1, 2012

(54) SELF-PROPELLED UNIT FOR ENDOSCOPE

(76) Inventors: Shinichi YAMAKAWA, Kanagawa (JP); Takayuki Nakamura, Kanagawa (JP); Masayuki Iwasaka, Kanagawa (JP)

(21) Appl. No.: 13/221,453

(22) Filed: Aug. 30, 2011

(30) Foreign Application Priority Data
Aug. 31, 2010 (JP) 2010-193839

(51) Int. Cl. A61B 1/00

(52) U.S. Cl. 600/114

(57) ABSTRACT

A self-propelled unit for an endoscope having a section of an elongated tube for entry in a body cavity is provided. A first ring sleeve is mounted around the elongated tube on a distal side in a rotatable manner. An endless track device is disposed around the first ring sleeve, having an outer annular surface, for turning around in contact with a wall of the body cavity, for propulsion in an axial direction of the elongated tube. A combination of a worm drive and an engagement roller is disposed on the first ring sleeve, for moving the endless track device.

A movable plate is contained in the endless track device, movable between open and closed positions, for coming farther from the elongated tube at least locally when in the open position than when in the closed position, in order to shift the endless track device away from the elongated tube.

### BACKGROUND OF THE INVENTION

1. Field of the Invention

The present invention relates to a self-propelled unit for an endoscope. More particularly, the present invention relates to a self-propelled unit capable of causing an endoscope to enter a body cavity smoothly, and preventing a proximal portion in a section of an elongated tube from locally contacting an endless track device.

2. Description Related to the Prior Art

An endoscope is used to diagnose a body cavity, such as a large intestine in a gastrointestinal tract. The endoscope has a flexible elongated tube for entry in the body cavity. The elongated tube includes a head assembly, a steering device and a flexible device. The head assembly is disposed at a distal end and has a short size. The steering device is present at a proximal end of the head assembly, and keeps the head assembly steerable in a desired direction. The flexible device extends from a proximal end of the steering device and has as large a length as 1-2 meters. In the head assembly, an imaging window is formed for image forming of an object of interest.

Manipulation of the endoscope is a difficult process, because the large intestine is a tortuous organ in a human body, and some body parts are very changeable in the position in the body, such as a sigmoid colon and a transverse colon. Learning the manipulation of the endoscope of the large intestine requires much experience and time. If a doctor is insufficiently skilled in the manipulation, physical load to the body of a patient will be very large. Various ideas are suggested for facilitating entry of the endoscope.

U.S. Ser. No. 2005/272,976 (corresponding to JP-A 2005-253892) discloses a self-propelled unit which has a peripheral portion mounted on a head assembly of the elongated tube of the endoscope. Ridges of a helical shape are formed on the peripheral portion, which is rotated to propel the head assembly in the body cavity.

U.S. Pat. Nos. 6,971,990 and 7,736,300 (corresponding to JP-A 2009-513250) disclose a self-propelled unit which includes a movable endless track device or crawler device or toroidal device. The endless track device is driven to turn around for the endoscope to travel mechanically. Force of propulsion is created by the endless track device contacting a wall of the large intestine, so as to guide the endoscope deeply in the body cavity.

In U.S. Ser. No. 2005/272,976, the ridges always contact a wall of the body cavity in a form of threads for advance in the body cavity. The peripheral portion must contact the wall. Thus, the self-propelled unit is suitable for a body cavity having a relatively small width. However, the peripheral portion must have a larger diameter for use in a large intestine which has a large width. It is very difficult to enter the self-propelled unit through the anus into the large intestine.

U.S. Pat. Nos. 6,971,990 and 7,736,300 disclose the self-propelled unit in which the endless track device has a very high flexibility, and can be deformed with a high degree of freedom for entry into the large intestine even if the endless track device has a somewhat large size. However, a support or housing of the endless track device longitudinally extends in the axial direction of the elongated tube. There is a problem in that the steering of the steering device is obstructed by the combined use of the guide assembly or the self-propelled unit, and that flexibility of the elongated tube may be lower. Accordingly, the manipulation may be more difficult.

### SUMMARY OF THE INVENTION

In view of the foregoing problems, an object of the present invention is to provide a self-propelled unit capable of causing an endoscope to enter a body cavity smoothly, and preventing a proximal portion in a section of an elongated tube from locally contacting an endless track device.

In order to achieve the above and other objects and advantages of this invention, a self-propelled unit for an endoscope having a section of an elongated tube for entry in a body cavity is provided, and includes a first ring sleeve mounted around a distal portion of the elongated tube. An endless track device is disposed around the first ring sleeve, has an end portion extending in a proximal direction further than the first ring sleeve, and has an outer annular surface, for endlessly turning around in an axial direction of the elongated tube by contact of the outer annular surface with a wall of the body cavity, to exert force of propulsion in the axial direction. Plural movable plates are contained in the endless track device, for supporting the endless track device by preventing looseness of the end portion of the endless track device, the movable plates having a proximal end portion being pivotally movable between open and closed positions, wherein the movable plates, when in the closed position, extend in the axial direction, and when in the open position, extend crosswise to the axial direction.

Furthermore, a second ring sleeve is contained in the endless track device, for keeping the endless track device movable in cooperation with the first ring sleeve.

Furthermore, a guide roller is secured to the proximal end of the movable plates in a rotatable manner, for contacting an inner surface of the endless track device.

The movable plates are secured to a proximal end of the second ring sleeve in a pivotally movable manner.

The endoscope includes a head assembly at a distal end, and a steering device for varying a direction of the head assembly. Furthermore, a retaining device is secured to the steering device, and connected with the first ring sleeve for retention.

The first ring sleeve is disposed with the steering device, and defines a clearance space with the steering device.

The second ring sleeve and the movable plates are disposed outside the steering device, and when the steering device is steered, at least one of the movable plates is moved toward the open position according to steering of the steering device.

Furthermore, an engagement roller is disposed on the first ring sleeve, for moving the endless track device by contacting the outer annular surface thereof. A pair of rollers are disposed on the second ring sleeve, for pressing the endless track device to the engagement roller.

In a preferred embodiment, a self-propelled unit for an endoscope having a section of an elongated tube for entry in a body cavity is provided, and includes a first ring sleeve mounted around the elongated tube on a distal side in a rotatable manner. An endless track device is disposed around the first ring sleeve, having an outer annular surface, for turning around in contact with a wall of the body cavity, for propulsion in an axial direction of the elongated tube. A driving device is secured to the first ring sleeve, for moving the endless track device. A movable plate is contained in the endless track device, movable between open and closed positions, for coming farther from the elongated tube at least locally when in the open position than when in the closed position, in order to shift the endless track device away from the elongated tube.

The movable plate, when in the open position, applies tension to the endless track device, to prevent a proximal end of the endless track device in the axial direction from loosening toward the elongated tube.

The second ring sleeve is constituted by a support ring, and the movable plate is secured to a proximal side of the support ring in a rotationally movable manner.

The elongated tube includes a steering device, disposed on the distal side, and having a variable direction. The movable plate is constituted by plural movable plates arranged in a circumferential direction in the second ring sleeve. When the steering device is bent in one steering direction, one of the movable plates corresponding to the steering direction moves to the open position, so as to facilitate steering of the steering device on a proximal side.

The driving device includes a sleeve of a worm shape supported on the first ring sleeve, and rotated around the elongated tube by external force. An engagement roller has teeth, supported on the second ring sleeve, meshed with the sleeve of the worm shape, for rotating to drive the endless track device.

Consequently, it is possible to cause an endoscope to enter a body cavity smoothly, and keep a steering device in the endoscope steerable readily, because the movable plate operates for shifting an endless track device away from the elongated tube.

### BRIEF DESCRIPTION OF THE DRAWINGS

The above objects and advantages of the present invention will become more apparent from the following detailed description when read in connection with the accompanying drawings, in which:

FIG. 1 is a plan, partially broken, illustrating an endoscope system;

FIG. 2 is a perspective view illustrating an endoscope and a self-propelled unit associated with the endoscope;

FIG. 3 is a vertical section illustrating the self-propelled unit;

FIG. 4 is an exploded perspective view illustrating a driving device in the self-propelled unit;

FIG. 5 is a vertical section illustrating the self-propelled unit in the course of steering of a steering device.

### DETAILED DESCRIPTION OF THE PREFERRED EMBODIMENT(S) OF THE PRESENT INVENTION

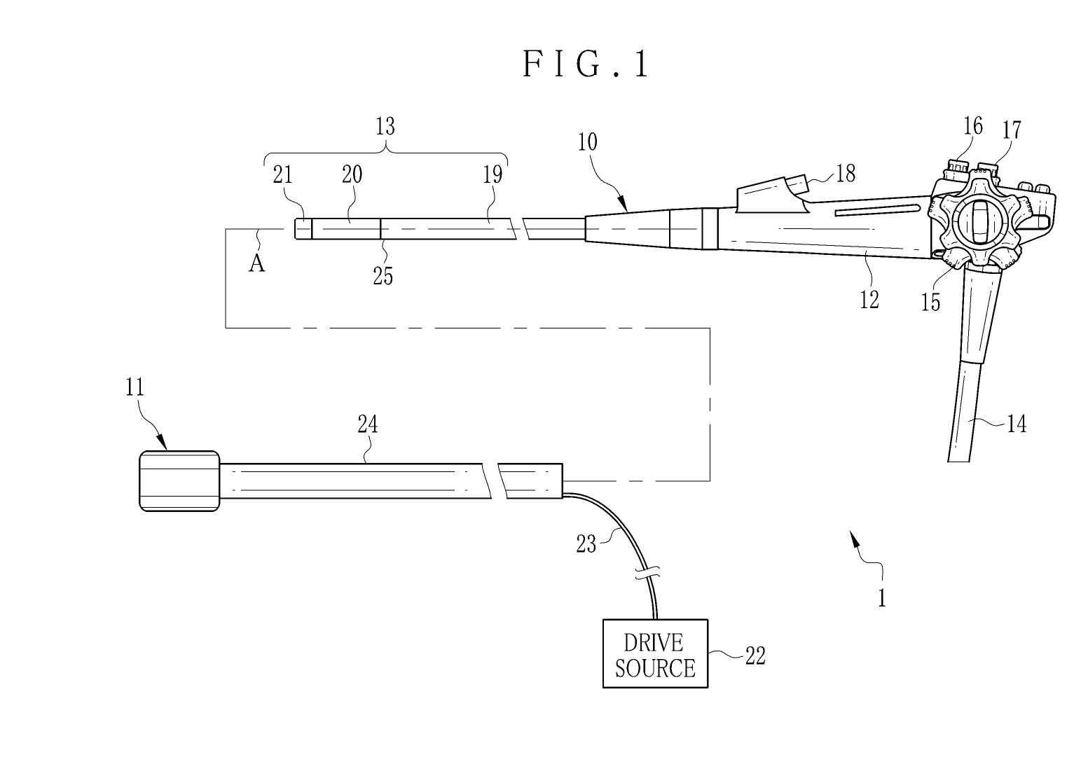

In FIG. 1, an electronic endoscope system 1 includes an electronic endoscope 10 and a self-propelled unit 11 or guide assembly. The endoscope 10 includes a handle device 12, and a section of an elongated tube 13 or guide tube extending from the handle device 12 for entry in a body cavity, for example, large intestine of a gastrointestinal tract. A universal cable 14 is connected with the handle device 12. Connectors or plugs are provided at an end of the universal cable 14 for connection with a light source apparatus (not shown) and a processing apparatus (not shown).

The handle device 12 includes steering wheels 15 for bending, an air/water button 16 and a suction button 17. The air/water button 16 is operable for supply of air or water through a distal end of the elongated tube 13. An instrument channel 18 is formed in the elongated tube 13 of the handle device 12 for receiving entry of a forceps, electrocautery device or other medical instrument.

The elongated tube 13 includes a flexible device 19, a steering device 20 and a head assembly 21 in a sequence in a distal direction from the handle device 12. The flexible device 19 has a length as great as 1-2 meters for reach of the head assembly 21 to an object of interest in a body cavity. The steering device 20 bends up and down and to the right and left in response to operation of the steering wheels 15 of the handle device 12. Thus, the head assembly 21 can be steered in a desired direction in the patient's body.

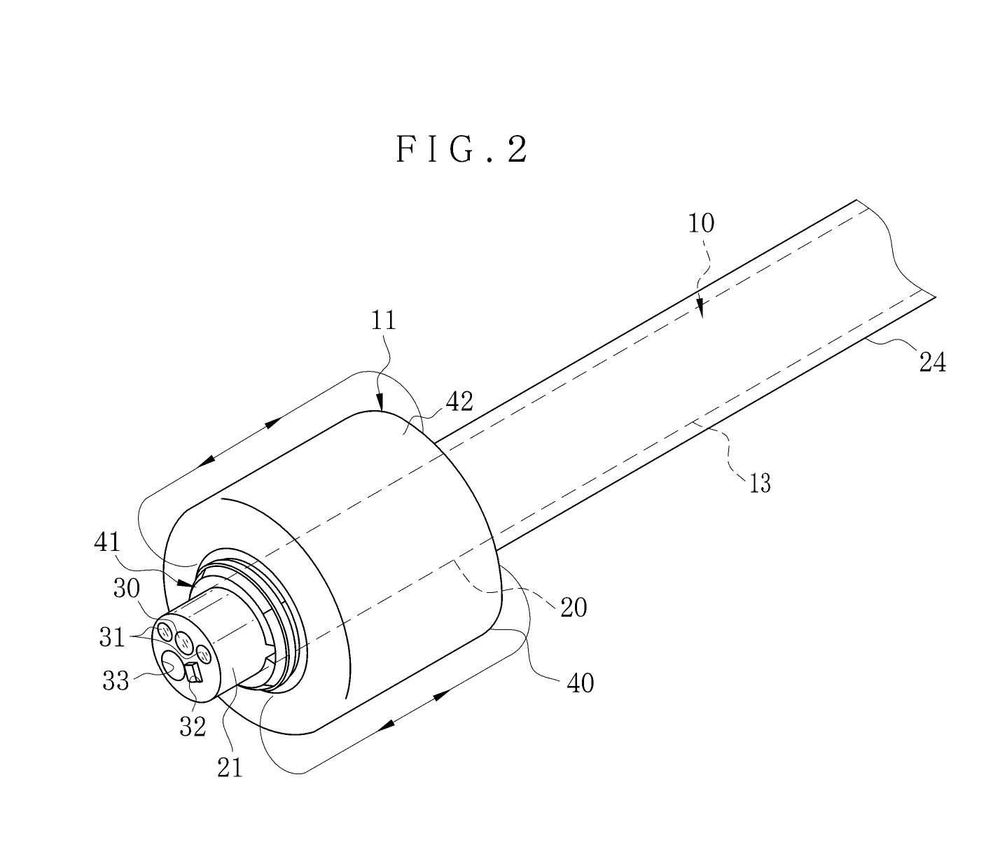

An imaging window 30 is formed in the head assembly 21 for imaging of a body part in the body. See FIG. 2. The head assembly 21 contains objective optics and an image sensor or solid-state image pickup device for imaging, such as CCD and CMOS image sensors. The image pickup device is connected to the processing apparatus by a signal line, which extends through the elongated tube 13, the handle device 12 and the universal cable 14. An object image of the body part is focused on a reception surface of the image pickup device, and is converted into an image signal. The processing apparatus processes the image signal from the image pickup device through the signal line by image processing, and obtains a video signal by conversion after the image processing. The object image is output and displayed on a monitor display panel (not shown) according to the video signal.

Various openings are formed in the head assembly 21 as illustrated in FIG. 2. Among those, a lighting window 31 passes illumination light from a light source apparatus toward the object of interest. An air/water nozzle 32 supplies air or water toward the imaging window from an air/water supply device in the light source apparatus in response to depression of the air/water button 16. An instrument opening 33 causes a distal end of a medical instrument from the instrument channel 18 to appear distally.

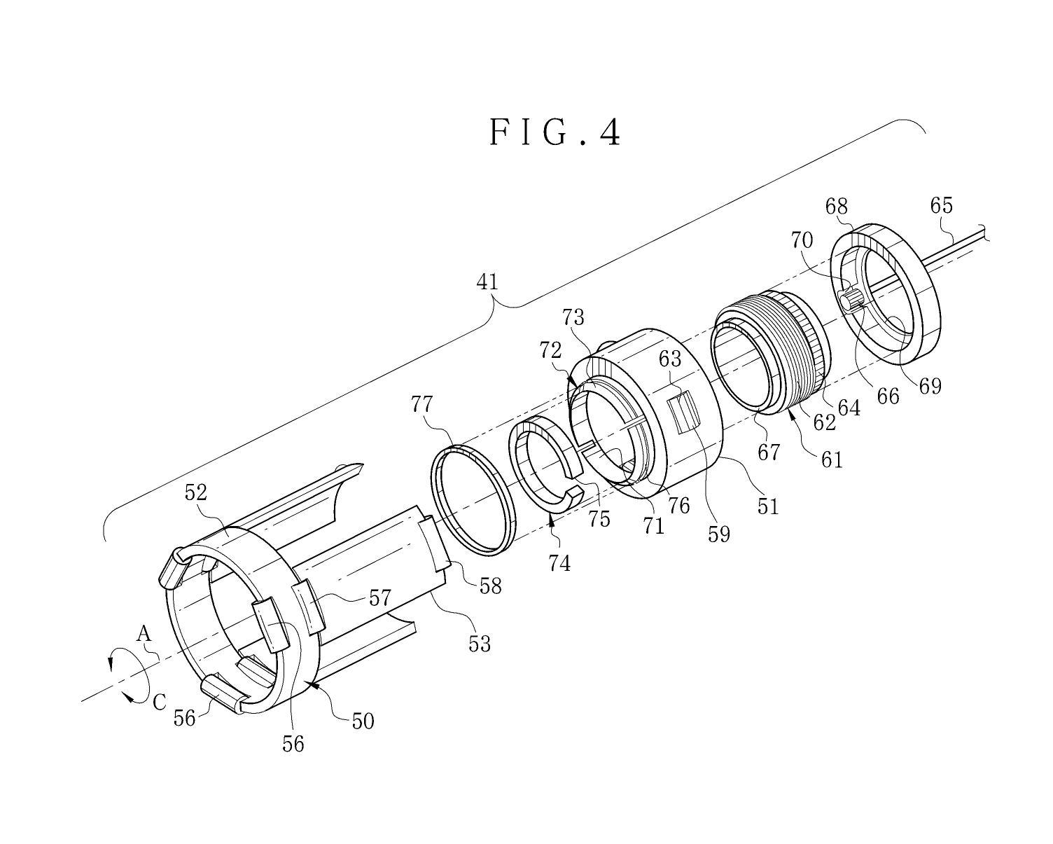

The self-propelled unit 11 is used with the endoscope 10 for assistance in moving the elongated tube 13 of the endoscope 10 in and out in through a body cavity. A drive source 22 or motor drives the self-propelled unit 11. A control wire 65 or torque wire of FIG. 4 is connected with the drive source 22, and transmits torque of rotation for driving the self-propelled unit 11. A protection sheath 23 extends with a full length of the control wire 65, and receives the control wire 65 for protection. The control wire 65 rotates in the protection sheath 23 when the drive source 22 is driven. Note that a coil structure may be used instead of the control wire 65.

An overtube 24 is used to cover the elongated tube 13, and is ready to expand and shrink in an axial direction A of the elongated tube 13. The protection sheath 23 of the control wire 65 is entered between the overtube 24 and the elongated tube 13.

A controller (not shown) controls the drive source 22. A button panel (not shown) is connected to the controller. The button panel includes a command button for inputting command signals for forward movement, backward movement and stop of the self-propelled unit 11, and a speed button for changing a moving speed of the self-propelled unit 11. Note that a control program can be prepared suitably for an object to be imaged. The drive source 22 can be actuated according to the control program without manipulating the button panel, so as to actuate the self-propelled unit 11 automatically.

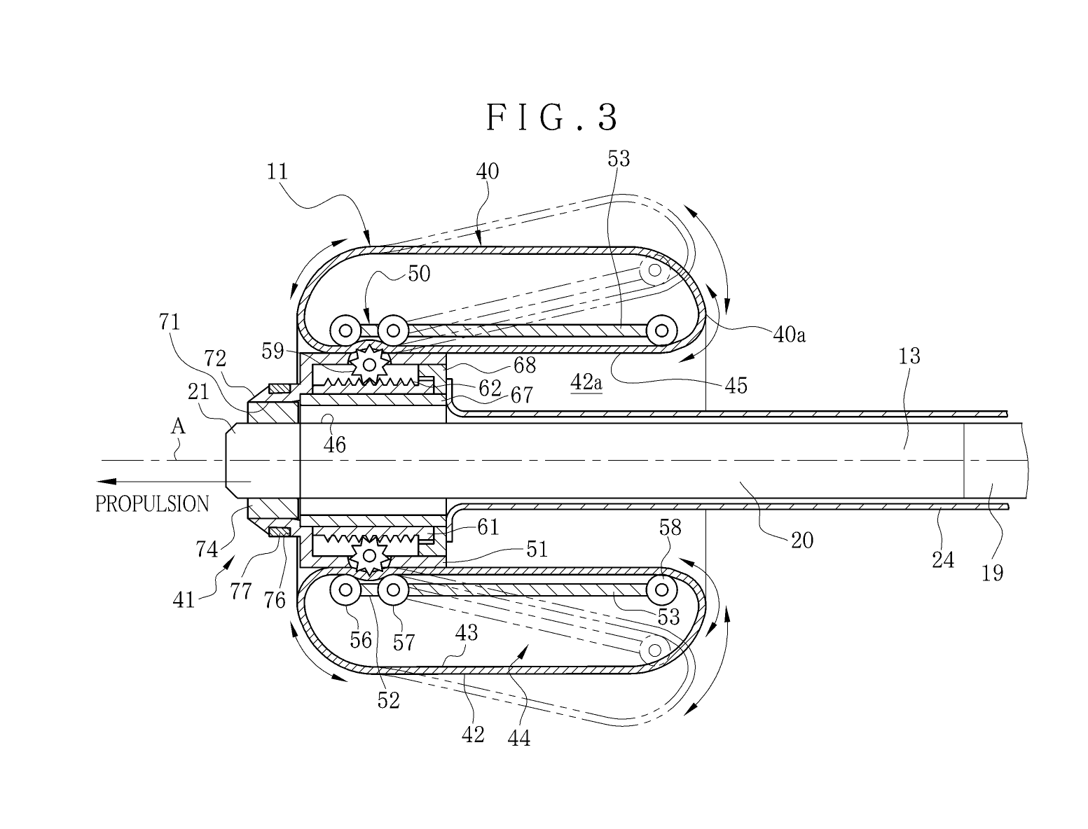

In FIG. 3, the self-propelled unit 11 is viewed in a section after division with an interval of 120 degrees. In FIGS. 2, 3 and 4, the guide assembly 11 includes a movable endless track device 40 or crawler device or toroidal device, and a sleeve device 41 or driving device or holder with a rotating mechanism. The endless track device 40 has a hollow shape with an outer annular surface 42, is movable on an endless track, and is formed from a biocompatible plastic material, such as polyvinyl chloride, polyamide resin, fluorocarbon resin and the like. The endless track device 40 includes the outer annular surface 42 and an inner surface 43.

A central opening 42a is defined in the outer annular surface 42 of the endless track device 40. A first ring sleeve 51 of the sleeve device 41 is disposed in the central opening 42a. An external portion of the outer annular surface 42 is moved in a first direction. An internal portion of the outer annular surface 42 at the central opening 42a is moved in a second direction reverse to the first direction. As a result, the endless track device 40 turns around the first ring sleeve 51 continuously from the inside to the outside of the central opening 42a. The external portion of the outer annular surface 42 exerts force by contacting a wall of a body cavity for propulsion of the elongated tube 13 in the axial direction A along the axis.

In FIG. 4, the sleeve device 41 is illustrated. The endless track device 40 is not shown. The sleeve device 41 has a second ring sleeve 50 and the first ring sleeve 51. There is a hollow space 44 in the endless track device 40. The second ring sleeve 50 is disposed in the hollow space 44. The first ring sleeve 51 is disposed between the elongated tube 13 of the endoscope 10 and the outer surface 42 of the endless track device 40. See FIG. 3. Also, it is possible to fill the hollow space 44 of the endless track device 40 with fluid of a biocompatible property, such as water, nitrogen gas or the like.

Specifically, the endless track device 40 is prepared in the following manner. At first, a plastic tube having two open ends with flexibility and elasticity is initially formed from a sheet or film of the above-described suitable material. The plastic tube is halfway inserted in a sleeve lumen of the second ring sleeve. Then a portion of the plastic tube outside the sleeve lumen is bent back externally and extended to cover the periphery of the second ring sleeve. A first side line of the inserted half of the plastic tube is opposed to a second side line of the bent half to the plastic tube, so that the halves are attached together along the first and second side lines by adhesion, welding or other suitable method. Finally, the toroidal shape of the endless track device 40 is obtained.

The first ring sleeve 51 has a cylindrical shape, and has a smaller range than the endless track device 40 in the axial direction A. The second ring sleeve 50 includes a support ring 52 or annular ring, and movable arms or plates 53. The support ring 52 has a smaller range than the first ring sleeve 51 in the axial direction A. The movable arms 53 are secured to an edge of the support ring 52 in a rotatable manner. An idler roller 57 or driven roller is provided, and keeps each one of the movable arms 53 rotatable on the support ring 52.

The movable arms 53 are rotationally movable between open and closed positions about each axis of the idler roller 57. When the movable arms 53 are in the closed position, their inner surface is flush with an inner surface of the support ring 52. A rear end portion 40a of the endless track device 40 of FIG. 3 projects from the second ring sleeve 50 toward the handle device 12. Each of the movable arms 53 prevents the rear end portion 40a from loosening toward the steering device 20 of the elongated tube 13. The movable arms 53, when in the open position, are rotated by approximately 15 degrees from the closed position, respectively facilitate steering of the steering device 20 without fail, and prevent the rear end portion 40a from breaking. A stopper mechanism (not shown) is incorporated between the support ring 52 and the movable arms 53 and near to the idler roller 57, and defines the open and closed positions of the movable arms 53.

When the movable arms 53 are in the closed position, a clearance space 45 of FIG. 3 is created between the steering device 20 and the rear end portion 40a of the endless track device 40.

An idler roller 56 or driven roller and the idler roller 57 are arranged in the axial direction A and secured to the second ring sleeve 50 in a rotatable manner. A guide roller 58 or end roller or idler roller is secured to each free end of the movable arms 53 on a proximal side, and disposed on a proximal side from the idler rollers 56 and 57. The guide roller 58 is disposed internally at the rear end portion 40a of the endless track device 40. The movable arms 53 are three arranged in a circumferential direction of the second ring sleeve 50 at an interval of 120 degrees. So are the idler rollers 56-58. Note that the movable arms 53 have a somewhat small width and are positioned with a large interval. However, an interval between the movable arms 53 in the rotational direction may be reduced in a range allowing the movable arms 53 to move reliably.

The idler roller 56 is disposed at a distal end for the support ring 52 of the second ring sleeve 50. The idler roller 57 is disposed at its proximal end, and also operates as a hinge mechanism where each movable arm 53 is secured to the support ring 52 in a rotatable manner, in short, in a swing door form. The idler rollers 56-58 contact the inner surface 43 of the endless track device 40, and are rotated by the turn around of the endless track device 40.

An engagement roller 59 or toothed roller or gear is disposed on a surface of the first ring sleeve 51 rotatably. The engagement roller 59 is constituted by three rollers arranged in a circumferential direction at an interval of 120 degrees. The engagement roller 59 is positioned in the middle of the first ring sleeve 51 in an axial direction A.

The engagement roller 59 is disposed between the idler rollers 56 and 57 of the second ring sleeve 50 in an assembled state of the self-propelled unit 11 so that the endless track device 40 is movable between the engagement roller 59 and the idler rollers 56 and 57. The engagement roller 59 contacts the outer surface 42 of the endless track device 40. A worm drive 61 or threaded sleeve or worm gear of a tubular shape rotates the engagement roller 59 to turn around the endless track device 40.

The worm drive 61 or threaded sleeve is contained in the first ring sleeve 51. In the axial direction A, a range of the worm drive 61 is slightly smaller than a range of the first ring sleeve 51. The worm drive 61 is in a symmetric form rotationally about the axis of the axial direction A. A worm thread 62 is disposed on the worm drive 61 helically about the axis. Engagement teeth 63 of the engagement roller 59 are meshed with the worm thread 62. Rotation of the worm drive 61 about the axis, namely in the circumferential direction C, is transmitted to the engagement roller 59. Preferably, lubricant is applied to the worm thread 62.

Spur gear teeth 64 are formed with a proximal end of the worm drive 61 or threaded sleeve, and arranged in the circumferential direction C. A pinion 66 is connected with the control wire 65, and meshed with the spur gear teeth 64. The pinion 66 is rotated by the control wire 65. The spur gear teeth 64 transmit the rotation of the pinion 66 to the worm drive 61, which rotates in the circumferential direction C.

There is a bearing sleeve 67 receiving insertion of the worm drive 61 or threaded sleeve. A distal edge of the bearing sleeve 67 is fitted on an inner surface of a distal end of the first ring sleeve 51. The elongated tube 13 of the endoscope 10 is introduced in the bearing sleeve 67. The bearing sleeve 67 has a smaller diameter than an inner diameter of the first ring sleeve 51 for the purpose of keeping a space for disposing the engagement roller 59 and the worm drive 61. The bearing sleeve 67 has a larger diameter than an outer diameter of the elongated tube 13 for the purpose of keeping a clearance space 46 (See FIG. 3) between an inner surface of the bearing sleeve 67 and an outer surface of the elongated tube 13.

A rear end ring 68 is fitted on a proximal end of the bearing sleeve 67, and keeps the worm drive 61 or threaded sleeve rotatable around the bearing sleeve 67, and keeps the worm drive 61 and the bearing sleeve 67 contained in the first ring sleeve 51. An outer diameter of the rear end ring 68 is equal to an inner diameter of the first ring sleeve 51. An opening 69 is formed in the rear end ring 68, and has a diameter equal to an outer diameter of the bearing sleeve 67. A cutout 70 is formed in the rear end ring 68, and contains the pinion 66 in a rotatable manner. A distal end of the control wire 65 is inserted in a hole (not shown) formed in the rear end ring 68, and connected with the pinion 66.

Thus, the worm drive 61 or threaded sleeve is driven by the pinion 66 and rotates about the axis of the axial direction A. The engagement roller 59 is rotated by the worm thread 62 of the worm drive 61. In response, the endless track device 40 turns around in a forward or backward direction according to one of rotational directions of the pinion 66 or the worm drive 61.

A retaining device 72 is disposed at a distal end of the first ring sleeve 51. A lumen 71 is formed through the first ring sleeve 51, and has a diameter slightly smaller than an outer diameter of the bearing sleeve 67. A range of the retaining device 72 in the axial direction A is sufficiently smaller than the first ring sleeve 51. A collet portion 73 or collet chuck device with plural collet segments or ridge segments constitutes the retaining device 72, and protrudes from an edge of the lumen 71 distally in the axial direction A. The collet segments in the collet portion 73 have an inclined surface with an inclination in an inward direction toward the distal side, and resiliently shift toward open and closed positions perpendicularly to the axial direction A.

A spacer 74 is a C ring, which is defined by a circular ring and a gap 75 formed in the ring to increase and decrease the crosswise size of the ring. An initial diameter of the spacer 74 is determined in consideration of an outer diameter of the elongated tube 13 of the endoscope 10 for fitting of the self-propelled unit 11. As the spacer 74 is deformable, plural types of the endoscope 10 for use with the self-propelled unit 11 can be combined by absorbing differences in the outer diameter of the elongated tube 13 even with the spacer 74 only.

The spacer 74 is secured to the inside of the retaining device 72 removably. To set the self-propelled unit 11 on the elongated tube 13 of the endoscope 10, at first the bearing sleeve 67, the spacer 74 and the retaining device 72 are caused to receive entry of the elongated tube 13. In a groove 76 which is formed in the retaining device 72, a rubber ring 77 or retaining ring is fitted. The collet portion 73 and the spacer 74 are firmly clamped by the rubber ring 77 in the inward direction, so that the self-propelled unit 11 is retained tightly on one connection portion of the elongated tube 13 of the endoscope 10, namely the head assembly 21 according to the embodiment.

Note that the connection portion of the elongated tube 13 as viewed in the axial direction has a size sufficient for keeping a stably retained state, for keeping flexibility of the elongated tube 13 of the endoscope 10, and for smoothing the bend of the steering device 20. The size is preferably equal to or more than 5 mm and equal to or less than 15 mm.

The operation of the endoscope system 1 is described now. At first, the overtube 24 is set to cover the elongated tube 13 of the endoscope 10. The self-propelled unit 11 is fitted on the head assembly 21 by use of the retaining device 72. To mount the self-propelled unit 11, the head assembly 21 is inserted in the bearing sleeve 67, the spacer 74 and the retaining device 72 in a sequence. Then the rubber ring 77 is fitted in the groove 76 of the retaining device 72.

After the overtube 24 and the self-propelled unit 11 are positioned and attached, the processing apparatus, light source apparatus and control apparatus are turned on for powering. Patient information and other required information is input. Then the elongated tube 13 of the endoscope 10 is entered in a body cavity of a patient's body.

After the head assembly 21 is advanced to a predetermined body part, for example, slightly short of a sigmoid colon, then the button panel is operated to turn on a power source for the drive source 22 of the self-propelled unit 11. Then a command signal for start is input with the button panel. The drive source 22 rotates the control wire 65 in a predetermined direction, so that rotation of the pinion 66 with the control wire 65 causes the worm drive 61 to rotate.

Rotation of the worm drive 61 or threaded sleeve is transmitted to the engagement roller 59 to turn around the endless track device 40. An outer portion of the endless track device 40 contacts a wall of a body cavity, to exert force of propulsion in the axial direction. The endless track device 40 of the self-propelled unit 11 pushes the wall of the body cavity from a distal side toward a proximal side, to propel the head assembly 21 in the distal direction along the wall.

When a command signal for a change is input by operating the button panel, the drive source 22 changes a rotational speed of the control wire 65. Thus, a moving speed of the self-propelled unit 11 is changed. When a command signal for return is input by operating the button panel, the drive source 22 causes the control wire 65 to rotate in a backward direction, to move the self-propelled unit 11 and the head assembly 21 backwards. When a command signal for a stop is input by operating the button panel, the drive source 22 stops to stop moving the self-propelled unit 11. It is possible to propel the head assembly 21 through the body cavity to an object of interest by suitably repeating those steps of the movement.

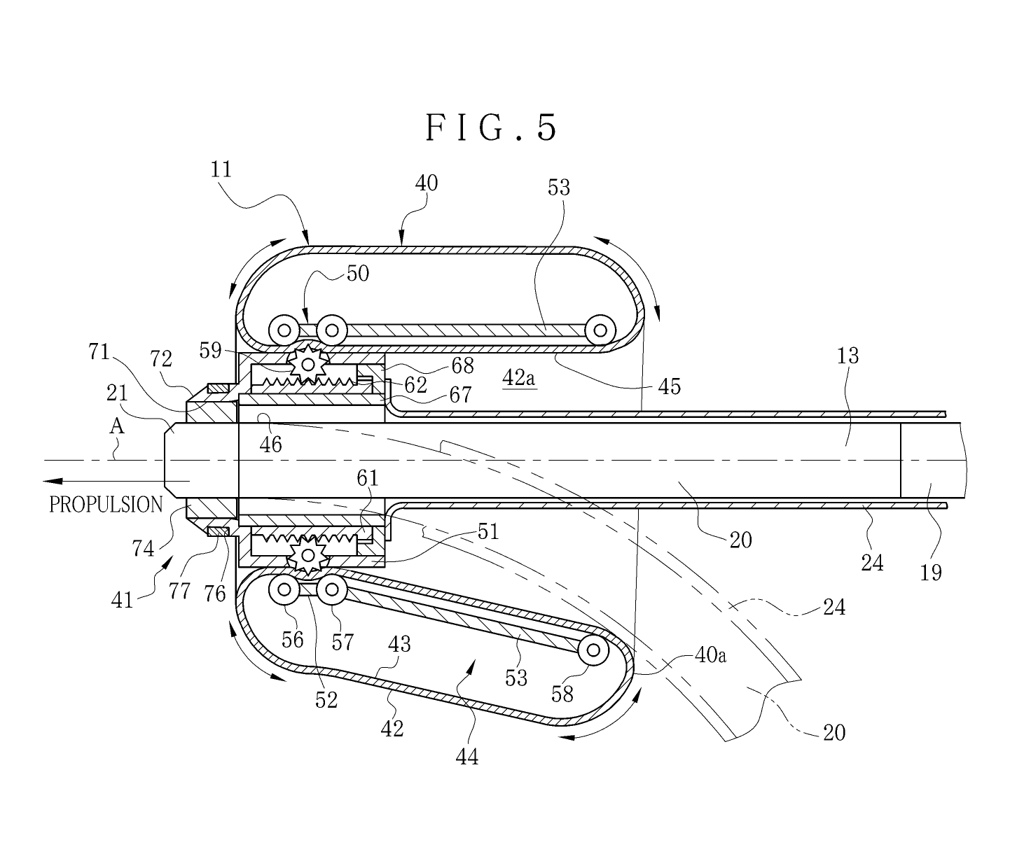

A doctor or operator suitably rotates the steering wheels 15 to steer the steering device 20 of the endoscope 10, and directs the head assembly 21 in a desired steering direction. In FIG. 5, an outer surface of the steering device 20 being steered presses the endless track device 40 to the outside in contact with the overtube 24. See the phantom line in FIG. 3. Thus, the guide roller 58 is pressed in an outward direction with the endless track device 40. The movable arm 53 of the second ring sleeve 50 is rotated from the closed position to the open position about an axis of the idler roller 57.

As the movable arm 53 rotates to the open position, the clearance space 45 is enlarged. Also, the clearance space 46 is kept by the bearing sleeve 67 and the spacer 74. It is possible to operate the steering device 20 for steering smoothly without blocking of the self-propelled unit 11 in as good a fashion as a state without the self-propelled unit 11. When the elongated tube 13 is in a straight form, the self-propelled unit 11 does not contact the elongated tube 13 except for a point at the retaining device 72. Thus, flexibility of the elongated tube 13 can be kept high by minimizing rigidity of the elongated tube 13 according to securing the self-propelled unit 11.

In the embodiment, a clearance space is created between an inner surface of the first ring sleeve 51 and an outer surface of the elongated tube. However, it is possible for the inner surface of the first ring sleeve 51 tightly to contact the outer surface of the elongated tube. The number of the movable arms may be different from three, for example, can be four or more. It is possible to modify the arrangement, number and the like of the idler rollers and engagement roller according to the embodiment.

In the embodiment, the idler roller 57 rotated by the turn around of the endless track device 40 operates also as a hinge mechanism for keeping each of the movable arms 53 movable rotationally. However, it is possible to dispose a hinge mechanism for the movable arms 53 discretely from the idler roller 57 for the endless track device 40. Such a hinge mechanism may be disposed on a distal side or proximal side from the idler roller 57.

In the above embodiment, the self-propelled unit is used with the endoscope for a medical use. Also, the guide assembly of the invention can be used with an endoscope for industrial use, an ultrasonic probe, or other instruments for imaging in a cavity.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A self-propelled unit for an endoscope having a section of an elongated tube for entry in a body cavity, comprising:
a first ring sleeve mounted around a distal portion of said elongated tube;
an endless track device, disposed around said first ring sleeve, having an end portion extending in a proximal direction further than said first ring sleeve, and having an outer annular surface, for endlessly turning around in an axial direction of said elongated tube by contact of said outer annular surface with a wall of said body cavity, to exert force of propulsion in said axial direction;
plural movable plates, contained in said endless track device, for supporting said endless track device by preventing looseness of said end portion of said endless track device, said movable plates having a proximal end portion being pivotally movable between open and closed positions, wherein said movable plates, when in said closed position, extend in said axial direction, and when in said open position, extend crosswise to said axial direction.

2. A self-propelled unit as defined in claim 1, further comprising a second ring sleeve, contained in said endless track device, for keeping said endless track device movable in cooperation with said first ring sleeve.

3. A self-propelled unit as defined in claim 2, further comprising a guide roller, secured to said proximal end of said movable plates in a rotatable manner, for contacting an inner surface of said endless track device.

4. A self-propelled unit as defined in claim 3, wherein said movable plates are secured to a proximal end of said second ring sleeve in a pivotally movable manner.

5. A self-propelled unit as defined in claim 4, wherein said endoscope includes a head assembly at a distal end, and a steering device for varying a direction of said head assembly;
further comprising a retaining device, secured to said steering device, and connected with said first ring sleeve for retention.

6. A self-propelled unit as defined in claim 5, wherein said first ring sleeve is disposed with said steering device, and defines a clearance space with said steering device.

7. A self-propelled unit as defined in claim 5, wherein said second ring sleeve and said movable plates are disposed outside said steering device, and when said steering device is steered, at least one of said movable plates is moved toward said open position according to steering of said steering device.

8. A self-propelled unit as defined in claim 7, further comprising:
an engagement roller, disposed on said first ring sleeve, for moving said endless track device by contacting said outer annular surface thereof;
a pair of rollers, disposed on said second ring sleeve, for pressing said endless track device to said engagement roller.
